(19) United States
(12) Patent Application Publication
(10) Pub. No.: US 2025/0064714 A1
(43) Pub. Date: Feb. 27, 2025

(54) COSMETIC COMPOSITION INCLUDING ETHOXYLATED MATERIALS TO IMPROVE PHYSICAL STABILITY OF POLYMERIC EMULSIONS

(71) Applicant: L'OREL, Paris (FR)

(72) Inventor: Nicholas David STEBBINS, Clark, NJ (US)

(21) Appl. No.: 18/456,242

(22) Filed: Aug. 25, 2023

(51) Int. Cl. A61K 8/92; A61K 8/06

(52) U.S. Cl. CPC A61K 8/92; A61K 8/062; A61K 2800/34; A61K 2800/48; A61K 2800/49; A61K 2800/51

(57) ABSTRACT

A fluid emulsion cosmetic composition includes a direct oil in water emulsion that includes at least one synthetic polymer or crosspolymer, at least one thickening polysaccharide, at least one ethoxylated agent having an HLB in a range from 8-14, at least one active selected from water soluble skin care actives, oil soluble skin care actives, and combinations thereof, water, and at least one oil. The composition is free of silicones. The fluid emulsion cosmetic composition maintains a fluid texture that does not break, become creamy or waxy or gellify, including over freeze thaw cycles, retaining a combination of water soluble and oil soluble actives present in an amount that is at least 10% by weight of the composition.

### FIELD OF THE INVENTION

The present invention is generally directed to a fluid emulsion cosmetic composition and method for manufacturing the same for providing benefits to the skin of a user, the fluid emulsion cosmetic composition including a combination of water soluble and oil soluble actives in a fluid emulsion for conferring benefits to the user's skin and having a favorable textural aesthetic that is stable from breaking of the emulsion in a light texture that is not creamy or waxy. Benefits of the fluid emulsion cosmetic composition include, but are not limited to, inclusion of >10% of a combination of water soluble and oil soluble actives with no loss in physical stability.

### BACKGROUND OF THE INVENTION

Cosmetic compositions in the form of emulsions that are stabilized only with polymers, as opposed to a combination of traditional emulsifiers and polymers combined, or traditional emulsifiers alone, can afford lightweight, fluid, gel-like textures with greater ease and this type of sensory is often well appreciated by many consumers. However, when oil soluble actives are employed together with water soluble actives, an emulsion type system is needed rather than purely aqueous serum or gel components which cannot support oil soluble actives. Thus, there is a need to have sensorial, yet stable emulsion-based serums which contain both water soluble and oil soluble actives. Many traditional emulsifiers are solid or waxy raw materials that need to be heated up to melt during processing, which expends significant energy in the heating and cooling process. Additionally, waxy emulsifiers often increase the viscosity and sensorial creaminess or richness of the formula, which is not appropriate or desirable to consumers who seek lighter, thinner products like toners, serums, and lotions. Unfortunately, without the use of traditional emulsifiers, emulsion stability is difficult to achieve. In the absence of an emulsifier which can surrounding the entire oil phase and preventing emulsion coalescence, the only raw material present to prevent instability is the polymer itself, which can prove insufficient. These polymers can emulsify or disperse the oil droplets in a gel-like matrix.

Thus, there is a need to provide a composition that includes raw materials that are not thick wax emulsifiers and can improve emulsion stability of polymer-stabilized formulas without significantly altering the sensory or galenic (i.e., not turning a serum into lotion or cream) of the base formula.

This disclosure provides a composition that employs ethoxylated raw materials to increase the physical stability of polymer-stabilized emulsions. Additionally, for naturality and renewability reasons, the fluid emulsion cosmetic composition is silicone-free. The absence of silicone provides a derivative benefit of permitting a greater breadth and concentration of oil soluble actives, including oil soluble actives that have poor solubility in pure silicones.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In various embodiments, a composition is provided for use according to the invention that includes a direct fluid emulsion composition and method for manufacturing the same. The disclosed fluid emulsion cosmetic composition maintains a fluid texture that does not break, become creamy or waxy or gellify, including over freeze thaw cycles, retaining actives present up to or beyond 10% of a combination of water soluble and oil soluble actives.

Provided in a first embodiment is a fluid emulsion cosmetic composition comprising: a direct oil in water emulsion comprising at least one synthetic polymer or crosspolymer, at least one thickening polysaccharide, at least one ethoxylated agent having an HLB in a range from 8-14, at least one active selected from water soluble skin care actives, oil soluble skin care actives, and combinations thereof, water, and at least one oil, wherein the composition is free of silicones.

In the various embodiments, the ethoxylated agent is in a liquid form at room temperature (“RT”=^{˜}25° C.).

Provided in another embodiment is a fluid emulsion cosmetic composition comprising: a direct oil in water emulsion comprising at least one synthetic polymer or crosspolymer present in a range from about 0.15% to about 1.5%, at least one thickening polysaccharide present in a range from about 0.05% to about 2%, at least one ethoxylated agent having an HLB in a range from 8-14 and present in a range from about 0.5% to about 5%, at least one active selected from water soluble skin care actives, oil soluble skin care actives, and combinations thereof, each active present in a range from about 1% to about 20%, water, and at least one oil present in a range from about 2% to about 4%, all amounts by weight, based on the weight of the fluid emulsion cosmetic composition, wherein the composition is free of silicones.

Provided in another embodiment is a fluid emulsion cosmetic composition comprising: a direct oil in water emulsion comprising at least one synthetic polymer or crosspolymer selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof, at least one thickening polysaccharide selected from the group consisting of Sclerotium gum, xanthan gum, cellulose gum, locust bean gum, carrageenan, and combinations thereof, at least one ethoxylated agent having an HLB in a range from 8-14, at least one active selected from water soluble skin care actives, oil soluble skin care actives, and combinations thereof, water, and at least one oil selected from the group consisting of pentaerythrityl tetraisostearate, c15-19 alkane, squalane, wherein the composition is free of silicones.

Provided in another embodiment is a fluid emulsion cosmetic composition comprising: a direct oil in water emulsion comprising at least one synthetic polymer or crosspolymer comprising acrylates/C10-30 alkyl acrylate crosspolymer, at least one thickening polysaccharide comprising xanthan gum, at least one ethoxylated surfactant comprising one of laureth-4 and PEG-7 glyceryl cocoate, a combination of water soluble and oil soluble actives comprising sodium thiosulfate, arginine, sodium hyaluronate, dipotassium glycyrrhizate, capryloyl salicylic acid, carnosine, hydroxyacetophenone, niacinamide, citric acid, caffeine, glycine-based skin conditioning agent, water and water soluble solvents comprising glycerin, alcohol denatured, butylene glycol, and pentylene glycol, and a combination of oils comprising pentaerythrityl tetraisostearate, c15-19 alkane, and squalane, wherein the composition is free of silicones.

Provided in another embodiment is a method of forming a fluid emulsion cosmetic composition as a direct oil in water emulsion, the method comprising providing at least one synthetic polymer or crosspolymer, at least one thickening polysaccharide, at least one ethoxylated agent having an HLB in a range from 8-14 that is liquid at room temperature, at least one active selected from water soluble skin care actives, oil soluble skin care actives, and combinations thereof, water, and at least one oil, wherein the composition is free of silicones, wherein the method includes blending all ingredients at room temperature without the application of heat.

In the various embodiments, the fluid emulsion cosmetic composition has a serum galenic that is stable from emulsion breaking and separation under freeze thaw conditions.

In the various embodiments, the fluid emulsion cosmetic composition maintains a fluid texture that does not break, become creamy or waxy or gellify, including over freeze thaw cycles, retaining a combination of water soluble and oil soluble actives present in an amount that is at least 10% by weight of the composition.

In some embodiments, the synthetic polymer or crosspolymer is present in a range from about 0.15% to about 1.5%, at least one thickening polysaccharide is present in a range from about 0.05% to about 2%.

In some embodiments, the combination of polymeric thickeners in the fluid emulsion cosmetic composition includes acrylates/C10-30 alkyl acrylate crosspolymer and xanthan gum.

In some embodiments, the at least one ethoxylated agent is one of laureth-4 and PEG-7 glyceryl cocoate.

In some embodiments, the at least one ethoxylated agent is one of laureth-4 and PEG-7 glyceryl cocoate, each present in a range from about 0.2% to about 5%, by weight of the fluid emulsion cosmetic composition.

In some embodiments, actives may include sodium thiosulfate, arginine, sodium hyaluronate, dipotassium glycyrrhizate, capryloyl salicylic acid, carnosine, hydroxyacetophenone, niacinamide, citric acid, caffeine, glycine-based skin conditioning agent, or a combination thereof, each present in a range from about 0.05% to about 10%, by weight of the composition.

In some embodiments, the actives include a combination of water soluble and oil soluble actives, the combination present in an amount that is at least 10% by weight of the composition.

In some embodiments, water is present in a range from about 60% to about 70%, by weight of the composition, and the fluid emulsion cosmetic composition includes water-soluble solvents that include glycerin, alcohol denatured, butylene glycol, and pentylene glycol, each present in a range from about 2% to about 4%, by weight of the composition.

In some embodiments, the at least one oil is selected from the group consisting of pentaerythrityl tetraisostearate, c15-19 alkane, squalane, and combinations thereof.

In some embodiments, the at least one oil is present in a range from about 0.5% to about 3, by weight of the fluid emulsion cosmetic composition.

In some embodiments, the at least one oil includes a combination of pentaerythrityl tetraisostearate, c15-19 alkane, and squalane, each present in a range from about 0.5% to about 3, by weight of the fluid emulsion cosmetic composition.

In some embodiments, the at least one synthetic polymer or crosspolymer is present in a range from about 0.15% to about 1.5%, the at least one thickening polysaccharide is present in a range from about 0.05% to about 2%, the at least one ethoxylated agent having an HLB in a range from 8-14 is present in a range from about 0.5% to about 5%, each active is present in a range from about 1% to about 20%, the at least one oil is present in a range from about 2% to about 4%, all amounts by weight, based on the weight of the fluid emulsion cosmetic composition.

In some embodiments, the least one synthetic polymer or crosspolymer is selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof, the least one thickening polysaccharide is selected from the group consisting of Sclerotium gum, xanthan gum, cellulose gum, locust bean gum, carrageenan, and combinations thereof, the at least one ethoxylated agent having an HLB in a range from 8-14 is selected from laureth-4 and PEG-7 glyceryl cocoate, the at least one active is selected from water soluble skin care actives, oil soluble skin care actives, and combinations thereof, water, and the at least one oil is selected from the group consisting of pentaerythrityl tetraisostearate, c15-19 alkane, squalane, and combinations thereof.

In some embodiments, the actives include a combination of water soluble and oil soluble actives, the combination present in an amount that is at least 10% by weight of the composition.

In another embodiment, provided is fluid emulsion cosmetic composition comprising: a direct oil in water emulsion comprising at least one synthetic polymer or crosspolymer comprising acrylates/C10-30 alkyl acrylate crosspolymer, at least one thickening polysaccharide comprising xanthan gum, at least one ethoxylated surfactant comprising one of laureth-4 and PEG-7 glyceryl cocoate, a combination of water soluble and oil soluble actives comprising sodium thiosulfate, arginine, sodium hyaluronate, dipotassium glycyrrhizate, capryloyl salicylic acid, carnosine, hydroxyacetophenone, niacinamide, citric acid, caffeine, glycine-based skin conditioning agent, water and water soluble solvents comprising glycerin, alcohol denatured, butylene glycol, and pentylene glycol, and a combination of oils comprising pentaerythrityl tetraisostearate, c15-19 alkane, and squalane, wherein the composition is free of silicones.

The fluid emulsion cosmetic composition according to claim 17, wherein the fluid emulsion cosmetic composition maintains a fluid texture that does not break, become creamy or waxy or gellify, including over freeze thaw cycles, retaining the combination of water soluble and oil soluble actives present in an amount that is at least 10% by weight of the composition.

In another embodiment, provided is a method of forming a fluid emulsion cosmetic composition as a direct oil in water emulsion, the method comprising providing at least one synthetic polymer or crosspolymer, at least one thickening polysaccharide, at least one ethoxylated agent having an HLB in a range from 8-14 that is liquid at room temperature, at least one active selected from water soluble skin care actives, oil soluble skin care actives, and combinations thereof, water, and at least one oil, wherein the composition is free of silicones, wherein the method includes blending all ingredients at room temperature without the application of heat.

In some embodiments, the actives include a combination of water soluble and oil soluble actives, the combination present in an amount that is at least 10% by weight of the composition.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

In the context of the invention, a direct emulsion is an oil in water emulsion.

“Keratinous substrate” and “keratinous tissue” each includes but is not limited to skin, hair, and nails.

“Natural” or “Nature-based” as used herein means and refers to cosmetically acceptable materials and components that are one or more of directly obtained from nature, are obtained from nature with minimal processing, and are derivatives of materials that are obtained from nature. Fluid emulsion cosmetic composition according to the instant disclosure are, in some embodiments, up to 99.11% natural, or “Highly natural” or “All Natural” which means that all carbon atoms of intentionally added ingredients are from natural sources per ISO 16128.

“Skin” as used herein means and refers to skin materials containing keratin such as facial and body skin, scalp, eyebrows, and lips.

“Stable” and “Stability” as used herein means that the fluid emulsion cosmetic composition does not show any signs of significant changes in one or more of color, odor, pH, or viscosity, or breaking/separation of the emulsion or excessive thickening to a creamy, waxy or gel texture, watery texture or gellification of the cosmetic composition, at room temperature and at temperatures in the range from about 25° C. to about 45° C. and remains stable over freeze thaw cycles from −20° C. to 20° C. for up to 10 days.

As used herein, the term “instability” refers to failure of a composition to remain fluid or serum-like and either demonstrate breaking/separation of the emulsion or excessive thickening to a creamy, waxy or gel texture, watery texture or gellification of the cosmetic composition. Instability can occur in compositions made outside of the scope of the disclosure (i.e., compositions lacking the disclosed polymer and ethoxylated agents), and compositions including amounts of polymeric thickener in excess of the amounts according to the disclosure, or conventional emulsifiers, thick emollients, and waxes.

The terms “Exclude,” “Free” and “Essentially Free” means that no reliably measurable excluded material is present in the fluid emulsion cosmetic composition, for example, an excluded gellan and waxes, plant butters, saturated fatty alcohols/acids above C16, silicones including silicone oils/emollients and silicone emulsifiers, including dimethicone, cyclomethicone, polysilicone-11, phenyl trimethicone, trimethylsilylamodimethicone, and stearoxytrimethylsilane, and other silicone oils, silicone emulsifiers, and silicone elastomers, petrochemicals, including benzalkonium chloride, isododecane, isohexadecane, sulfate based surfactants, emulsifiers selected from those with a C10-C20 fatty alcohol or acid hydrophobe condensed with from about 2 to about 100 moles of ethylene oxide or propylene oxide per mole of hydrophobe, C2-C10 alkyl phenols condensed with from 2 to 20 moles of alkylene oxide, mono- and di-fatty acid esters of ethylene glycol, fatty acid monoglyceride, sorbitan, mono- and di-C8-C20 fatty acids, polyoxyethylene sorbitan, alkyl polyglycosides and saccharide fatty amides (e.g. methyl gluconamides), alkyl ether sulfate and sulfonates, alkyl sulfates and sulfonates, alkylbenzene sulfonates, alkyl and dialkyl sulfosuccinates, C8-C20 acyl isethionates, C8-C20alkyl ether phosphates, alkylethercarboxylate. Some specific emulsifiers that are lacking from the composition include PEG-100 Stearate, PEG-20 Stearate, and other esters of Poly(EthyleneGlycol), Sucrose Stearate and other emulsifiers based on sugar esters, Glyceryl Stearate and other glycerol esters, Disodium Ethylene Dicocamide PEG-15 Disulfate, Sodium Steroyl Glutamate and other fatty amides, Steareth-100 and other fatty ethers, or other excluded material as described herein. The term “essentially free” means that, while it is preferred that no excluded material is present in the fluid emulsion cosmetic composition, it is possible to have very small amounts of the excluded material in the fluid emulsion cosmetic composition of the invention, provided that these amounts do not materially affect the advantageous properties of the fluid emulsion cosmetic composition. In particular, “essentially free” means that excluded material can be present in the fluid emulsion cosmetic composition at an amount of less than 5%, or less than 4%, or less than 3%, or less than 2%, or less than 1%, or less than 0.1% by weight, based on the total weight of the fluid emulsion cosmetic composition.

As shown herein, the inventors have provided the fluid emulsion cosmetic composition in the form of a direct oil in water emulsion that includes water and oily phases and includes at least one synthetic polymer or crosspolymer, at least one thickening polysaccharide, at least one ethoxylated agent having an HLB in a range from 8-14, at least one active selected from water soluble skin care actives, oil soluble skin care actives, and combinations thereof, water, and at least one oil, wherein the composition is free of silicones. The fluid emulsion cosmetic composition maintains a fluid texture that does not break, become creamy or waxy or gellify, including over freeze thaw cycles, retaining a combination of water soluble and oil soluble actives present in an amount that is at least 10% by weight of the composition.

The fluid emulsion cosmetic composition may also include one or more additional ingredients selected from, but not limited to, phenylethyl resorcinol, chelating agents, other skin active components, antimicrobials and preservatives, fillers, antioxidants, fragrances, pearlescent agents, odor absorbers, coloring materials, essential oils, vitamins, and combinations of these.

The inventors sought to develop a solution for stabilizing fluid emulsions, for example thin, serum type composition, that contain high amounts of oil soluble and water-soluble actives (equal to or greater than 10%) without breaking of the emulsion or excessive and undesirable thickening or creaminess. The inventors have surprisingly found that ethoxylated ingredients, including ethoxylated fatty alcohols and ethoxylated fatty acids, represent a class of raw materials (herein referred to as “ethoxylated agents”) that can provide the advantage of maintaining a fluid, serum-like consistency in absence of silicones, with an added benefit of enabling incorporation of a wide range of actives (water and/or oil soluble) that may have resistance to solubility in silicones. In the general cosmetic arts, ethoxylated fatty alcohols and ethoxylated fatty acids are known as emulsifiers or as solubilizers and are characterized by the presence of a hydrophobic alky fatty alcohol/acid portion and a hydrophilic ethylene oxide portion. Existing commercial products that include silicones or trace amounts of ethoxylated agents tend to carry relatively low amounts of oil and water-soluble actives, and other products that include higher amounts of both water and oil soluble actives are thick creams or gel creams that also contain waxy materials. The instant invention solves the need of providing a light, thin emulsion that carries high amounts of water and oil soluble actives that is stable to freeze thaw conditions. It is posited that ethoxylated surfactant stabilizes the oil phase with the polymeric thickener gum without further thickening, thus preserving the fluid serum-like consistency of the emulsion. Specific examples of these ethoxylated agents, as shown in the examples, include laureth-4 and PEG-7 glyceryl cocoate.

This invention describes a way to stabilize a fluid polymer-stabilized, emulsion-based serum without adding waxiness or high viscosity through selection of ethoxylated emulsifiers. Other polymers can be more electrolyte intolerant or thicken in such a way that adds heaviness and body or thicken excessively to turn a light serum into a lotion or cream. I have identified the key RM attributes (liquid, HLB value, presence of ethoxylation) that affords good stability in comparison with other RMs that do not fall within those attributes. The ethoxylated emulsifiers showed greatly improved freeze/thaw stability verses the base and the polyglyceryl emulsifiers (as shown by microscope images).

While it may be expected in the art that the HLB of a surfactant can be a main driver for emulsion stability when all other ingredients are kept constant, the inventors found unexpectedly that this is not the case with respect to the instant inventive composition. As shown herein, it was found that that laureth-4, PEG-7 glyceryl cocoate, and polyglyceryl-6 dicaprate all have very similar HLB values. However, only the former 2 showed the large improvement in stability. Therefore, the HLB value is not the main driver of the stability, which is an unexpected attribute of this system. In addition, this can be done without the use of silicones (emollients or emulsifiers). This system can easy hold large quantities >10% of water-soluble and oil-soluble actives with no loss in physical stability.

The performance(s) of the inventive composition compared to benchmark lacking the claimed combination of ingredients is better than benchmark, including improved stability over formulations that do not have ethoxylated emulsifiers, improved sensory over formulations that would need to stabilize using high amounts polymer or with waxes, improved sustainability over formulations that use silicones.

The fluid emulsion cosmetic composition is sustainable when compared with silicone-containing light serums, wherein in the inventive composition a low level (0.15%) of synthetic polymer is needed, and the balance of the fluid emulsion cosmetic composition can include completely renewable and biodegradable components including gum thickeners, actives, solvents, oils, and other natural or nature-based ingredients.

In accordance with the various embodiments, it is contemplated that some or all of following ingredients may be excluded from the fluid emulsion cosmetic compositions used according to the invention: gellan, waxes, plant butters greater than about 5%, by weight, based on the total weight of the fluid emulsion cosmetic composition, and saturated fatty alcohols/acids above C16 present in amounts greater than about 5%, by weight, based on the total weight of the fluid emulsion cosmetic composition, and silicones including silicone oils/emollients and silicone emulsifiers. Other ingredients that may be excluded are petrochemicals and sulfate-based surfactants.

Accordingly, in various embodiments, the fluid emulsion cosmetic composition is provided according to the following detailed description.

### Polymeric Thickeners (Polymers)

In accordance with the disclosure, the fluid emulsion cosmetic composition includes a combination of polymeric thickeners that include at least one synthetic polymer or crosspolymer, and at least one thickening polysaccharide.

In some embodiments, the least one synthetic polymer or crosspolymer is selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof.

In some embodiments, the at least one thickening polysaccharide is selected from the group consisting of gums, cellulose derivatives, and combinations thereof.

In some embodiments, the at least one thickening polysaccharide is a gum selected from the group consisting of Sclerotium gum, xanthan gum, cellulose gum, locust bean gum, carrageenan, and combinations thereof.

In some embodiments, the combination of polymeric thickeners in the fluid emulsion cosmetic composition includes acrylates/C10-30 alkyl acrylate crosspolymer and xanthan gum.

In some embodiments, any one of the least one synthetic polymer or crosspolymer is present in a range from about 0.15% to about 1.5%, by weight, based on the total weight of the fluid emulsion cosmetic composition. In some embodiments, the total amount of the synthetic polymer or crosspolymer present in the fluid emulsion cosmetic composition is not more than about 2%, total, by weight, based on the total weight of the fluid emulsion cosmetic composition. In some embodiments, the total amount of the least one synthetic polymer or crosspolymer is at least about 0.1%, or at least about 0.15%, or about 0.6%, or about 0.8%, or about 1%, or about 1.2%, or about 1.5%, or any suitable combination, sub-combination, range, or sub-range thereof by weight, based on the weight of the fluid emulsion cosmetic composition. One of ordinary skill in the art, however, will appreciate that other ranges are within the scope of the invention.

In some embodiments, any one of the thickening polysaccharides is present in a range from about 0.05% to about 2%, by weight, based on the total weight of the fluid emulsion cosmetic composition. In some embodiments, the total amount of the thickening polysaccharide present in the fluid emulsion cosmetic composition is in an amount from about 0.03% to about 3%, or from about 0.05% to about 2%, or from about 0.2% to about 2%, or from about 0.3% to about 1.5%, or about 0.35%, or any suitable combination, sub-combination, range, or sub-range thereof by weight, based on the weight of the fluid emulsion cosmetic composition. One of ordinary skill in the art, however, will appreciate that other ranges are within the scope of the invention.

Thus, each one of polymeric thickeners, alone or in combination, is present in the fluid emulsion cosmetic composition, by weight, based on the total weight of the fluid emulsion cosmetic composition, as disclosed above, from about 0.05, 0.06, 0.07, 0.08, 0.09, 0.10, 0.20, 0.30, 0.40, 0.50, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, to about 3.0 percent, including increments and ranges therein and there between.

### Ethoxylated Agents (Ethoxylated Agents)

In accordance with the disclosure, a fluid emulsion cosmetic composition according to the disclosure includes at least one ethoxylated emulsifier agent in liquid form at ambient temperature in a range from about 20° C. to about 28° C. (in some embodiments referred to as room temperature, “RT”=˜ 25° C.) and has an HLB from about 8.5 to about 16.

The term “Hydrophilic-Lipophilic Balance” or “HLB,” refers to an empirical expression for the relationship of the hydrophilic and hydrophobic groups of an emulsifier. This term is well known to those skilled in the art. See, e.g., “The HLB system. A time-saving guide to Emulsifier Selection” (Pub: ICI Americas Inc., 1984) and US2006/0217283 at [0053].

In various embodiments, the at least one ethoxylated agent has an HLB that is in the range inclusive of from about 8 to about 14, for example the ethoxylated emulsifier has an HLB that is in a range from about 8.0, 8.1, 8.2, 8.3, 8.4, 8.5, 8.6, 8.7, 8.9, 9, 10, 11, 12, 13, to about 14, including increments of about 0.1 therein and therebetween.

In some embodiments, a fluid emulsion cosmetic composition includes at least one ethoxylated agent in liquid form at ambient temperature in a range from about 20° C. to about 28° C. and has an HLB from about 8 to about 14 selected from the group consisting of ethoxylated fatty alcohols, ethoxylated fatty acids, and combinations thereof.

In some embodiments, the at least one ethoxylated agent is selected from the group consisting of laureth-4, PEG-7 glyceryl cocoate, oleth-10, oleth-5, laureth-9, PEG-7 caprylic/capric glycerides, PEG-6 caprylic/capric glycerides, and combinations thereof.

In some embodiments, the at least one ethoxylated agent is one of laureth-4 and PEG-7 glyceryl cocoate.

In some embodiments, the fluid emulsion cosmetic composition includes one or more ingredients selected from the group consisting of ethoxylated agents, each present in a range from about 0.2% to about 5%, or from about 0.5% to about 2%, or not more than about 5%, by weight of the fluid emulsion cosmetic composition.

In some embodiments, the at least one ethoxylated agent can include any laureth-x, where x is from 4 to about 12.

In some embodiments, the at least one ethoxylated agent can include any oleth-x, where x is from 5 to about 12.

In some embodiments, the at least one ethoxylated agent can include any PEG-X laurate where X is from 4 to about 12.

In some embodiments, the at least one ethoxylated agent can include any PEG-X isostearate where X is from 5 to about 12.

In some embodiments, the at least one ethoxylated agent can include any PEG-X oleate where X is from 5 to about 12.

In some embodiments, any one of the at least one ethoxylated agent is present in a range from about 0.2% to about 5%, by weight, based on the total weight of the fluid emulsion cosmetic composition. In some embodiments, the total amount of the at least one ethoxylated agent present in the fluid emulsion cosmetic composition is not more than about 5%, total, by weight, based on the total weight of the fluid emulsion cosmetic composition. In some embodiments, the total amount of the at least one ethoxylated agent is at least about 0.4%, or at least about 0.5%, or at least about 0.6%, or at least about 0.8%, or at least about 1%, or at least about 1.2%, or at least about 1.5%.

The amount of the at least one ethoxylated agent present in the fluid emulsion cosmetic composition is provided in a range of from about 0.2% to about 5% by weight, or from about 0.5% to about 3% by weight, or from about 0.8% to about 1.5% or any suitable combination, sub-combination, range, or sub-range thereof by weight, based on the weight of the fluid emulsion cosmetic composition. One of ordinary skill in the art, however, will appreciate that other ranges are within the scope of the invention.

Thus, any one ethoxylated agent or a combination ethoxylated agents are present in the fluid emulsion cosmetic composition, by weight, based on the total weight of the fluid emulsion cosmetic composition, from about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, to about 5 percent, including increments and ranges therein and there between.

### Actives

In accordance with the disclosure, a fluid emulsion cosmetic composition according to the disclosure includes at least one water soluble active, at least one oil soluble active, or combination of water and oil soluble actives. In some embodiments, the fluid emulsion cosmetic composition according to the disclosure includes at least one water soluble active and at least one oil soluble active.

Generally, actives may be selected from among any known skin care actives. Actives may or may not include UV sunscreen actives. Actives may be selected from, for example, but not limited to: hydroxyacetophenone; niacinamide, vitamins, for example, vitamin A, beta carotene, tocopherol/vitamin E, panthenol, calcium pantothenate, retinol, resveratrol, vitamin C, niacinamide, hyaluronic acid, hydrolyzed hyaluronic acid, sodium hyaluronate, essential oils, antioxidants, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, phenolic terpenes, resveratrol, curcumin, pinoresinol, ferulic acid, hydroxytyrosol, cinnamic acid, caffeic acid, p-coumaric acid, baicalin (Scutellaria baicalensis root extract), mangiferin, pine bark extract (Pinus pinaster bark/bud extract), ellagic acid), escin, dipotassium glycyrrhizate, phenylethyl resorcinol, alpha hydroxy acids, and derivatives and combinations of the foregoing.

In some embodiments, actives may include sodium thiosulfate, arginine, sodium hyaluronate, dipotassium glycyrrhizate, capryloyl salicylic acid, carnosine, hydroxyacetophenone, niacinamide, citric acid, caffeine, glycine-based skin conditioning agent, or a combination thereof, each present in a range from about 0.05% to about 10%, by weight of the composition.

In some particular embodiments, the actives may include sodium thiosulfate present from about 0.15% to about 0.3% or about 0.25%, arginine present from about 1% to about 2% or about 1.5%, sodium hyaluronate present from about 0.05% to about 0.08% or about 0.07%, dipotassium glycyrrhizate present from about 0.1% to about 0.3% or about 0.2%, capryloyl salicylic acid present from about 0.25% to about 0.35% or about 0.3%, carnosine present from about 0.25% to about 0.35% or about 0.3%, hydroxyacetophenone present from about 0.25% to about 0.35% or about 0.3%, niacinamide present from about 5% to about 15% or about 10%, citric acid present from about 0.25% to about 0.7% or about 0.45% to about 0.5%, caffeine present from about 0.5% to about 1.5% or about 1%, glycine-based skin conditioning agent present from about 0.4% to about 0.6% or about 0.5%, or a combination thereof, and more generally, each present in a range from about 0.05% to about 15%, by weight of the composition, and in some embodiments in a total amount that is about 10% or more, or to abut 15% or to about 20%.

Although the actives are given as an example, it will be appreciated that other actives compatible with skin care applications known in the art may be used.

In accordance with the various embodiments, the amount of one or more actives, selected from water and oil soluble actives, alone or in combination, when present in the fluid emulsion cosmetic composition according to the disclosure can be present in a range from about 0.0001% to about 20%, or from 0.05% to about 10%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the fluid emulsion cosmetic composition. One of ordinary skill in the art, however, will appreciate that other ranges are within the scope of the invention.

Thus, any one or a combination of actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

### Solvents

### Water

In accordance with the various embodiments, water is present in the fluid emulsion cosmetic composition in a range from about 10% to about 95%, or from about 20% to about 85%, or from about 30% to about 80%, or from about 40% to about 75%, or from about 45% to about 65%, or any suitable combination, sub-combination, range, or sub-range thereof by weight, based on the weight of the fluid emulsion cosmetic composition. One of ordinary skill in the art, however, will appreciate that other ranges are within the scope of the invention.

Thus, water may be present by weight, based on the weight of the fluid emulsion cosmetic composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90 to about 95 weight percent, including increments and ranges therein and there between.

In some embodiments, water is present in a range from about 60% to about 70%, by weight of the composition.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene. The water phase may also comprise reconstituted thermal water, a water comprising trace elements such as zinc, copper, magnesium, etc., reconstituting the characteristics of a thermal water.

The pH of the fluid emulsion cosmetic composition is adjusted based on the amount of cosmetically acceptable acid to be in a range from about 3.5 to about 8.5, or from about 4.5 to about 7, or from about 5 to about 6 or from about 6 to about 7. The pH is adjusted to the desired value by addition of a base (organic or inorganic), for example sodium hydroxide, potassium hydroxide, or another suitable base, or combinations thereof.

### Water-Soluble Solvents

In accordance with some embodiments, the fluid emulsion cosmetic composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), organic solvents, polyols, glycols, or mixtures thereof.

In some embodiments according to the disclosure, when present, a water-soluble solvent may be selected from the group consisting of glycerin, alcohol denatured, butylene glycol, pentylene glycol, and combinations thereof.

In some embodiments, water-soluble solvents include glycerin, alcohol denatured, butylene glycol, and pentylene glycol, each present in a range from about 1% to about 10%, or from about 2% to about 4%, by weight of the composition. According to some such embodiments glycerin and alcohol denatured may be present at about 4% each, and butylene glycol and pentylene glycol may be present at about 2% each.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments the amount of the at least one water-soluble solvent, when present, is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the fluid emulsion cosmetic composition. One of ordinary skill in the art, however, will appreciate that other ranges are within the scope of the invention. In some embodiments, the fluid emulsion cosmetic composition includes more than one water soluble solvent, each water-soluble solvent present in an amount as set forth herein above, wherein each different water-soluble solvent may be present within one of the ranges selected from the ranges set forth herein above.

Thus, each one or combination of water-soluble solvents, when present, may be present by weight, based on the total weight of the fluid emulsion cosmetic composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Oily Phase: Oil

In accordance with the various embodiments, the fluid emulsion cosmetic composition includes at least one oil. The at least one oil is present in the oily phase. The oily phase also includes one or more oil soluble actives. In some embodiments, the oil is generally immiscible in water. The oil may be selected from hydrocarbons, silicones, fatty alcohols, glycols, and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof.

In some embodiments according to the disclosure, the at least one oil is selected from the group consisting of pentaerythrityl tetraisostearate, c15-19 alkane, squalane, and combinations thereof.

In some embodiments according to the disclosure, the at least one oil includes a combination of pentaerythrityl tetraisostearate, c15-19 alkane, and squalane, each present in a range from about 0.5% to about 3, by weight of the fluid emulsion cosmetic composition.

As used herein, oil refers to any nonpolar compound that is a liquid at 25° C. and is hydrophobic and lipophilic. Oil that is suitable for use herein may be volatile or non-volatile. The term “volatile oil” relates to oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10-3 to 300 mmHg). The term “non-volatile oil” relates to oil which remains on the skin or the keratin fiber, at room temperature and atmospheric pressure, for at least several hours and which has a vapor pressure of less than 10-3 mmHg (0.13 Pa).

In accordance with the disclosure, by way of non-limiting example, the one or more oil in the fluid emulsion cosmetic composition may be chosen from botanical and essential oils, such as Helianthus annuus Seed Oil, Lavandula angustifolia (lavender) Oil, Mentha piperita Oil, Rosmarinus officinalis (rosemary) Leaf Oil Pelargonium graveolens flower oil, Citrus aurantium dulcis (orange) peel oil, Menthe viridis (spearmint) leaf oil, Citrus aurantifolia (lime) oil, Melaleuca alternifolia (tea tree) leaf oil, Citrus grandis (grapefruit) peel oil, Citrus medica limonum (lemon) peel oil, rose flower oil, Eucalyptus globulus leaf oil, and combinations thereof.

### Hydrocarbon-Based Oils

The fluid emulsion cosmetic composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the fluid emulsion cosmetic composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, Eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the fluid emulsion cosmetic composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the fluid emulsion cosmetic composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the fluid emulsion cosmetic composition may comprise polar emollients that include those derived from C12-C50 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, alone or in combination as a blend of oils, may be present in the fluid emulsion cosmetic composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the fluid emulsion cosmetic composition. One of ordinary skill in the art, however, will appreciate that other ranges are within the scope of the invention. In some embodiments, the fluid emulsion cosmetic composition includes more than one oil, each oil present in an amount as set forth herein above, wherein each different oil (such as, for example, plant oils and extracts with oils) may be present within one of the ranges selected from the ranges set forth herein above.

Thus, each of the at least one oil or combination of oils is present by weight, based on the total weight of the fluid emulsion cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19 to about 20 percent, including increments and ranges therein and there between.

### Other Ingredients

In accordance with the various embodiments, the fluid emulsion cosmetic composition may further include additional ingredients, generally including, but not limited to, chelating agents, pH adjusters, skin actives, skin conditioning agents, including for example, glycine-based conditioning agents, humectants, antioxidants, plant extracts, plant oils and butters, fragrances, pearlescent agents, odor absorbers, coloring materials, essential oils, vitamins, antimicrobials and preservatives, and combinations of these. Non limiting examples of some of optional additional ingredients are provided herein below.

### Chelating Agents

In some embodiments, one or more other components, such as chelating agents can be present in the fluid emulsion cosmetic composition, in amounts from about 0.01% to about 2% by weight, from about 0.02% to about 1.5% by weight, from about 0.02% to about 1%, from about 0.02% to about 0.5%, and from about 0.2 to about 0.4%, or any suitable combination, sub-combination, range, or sub-range thereof by weight, based on the weight of the fluid emulsion cosmetic composition. In some exemplary embodiments, chelating agents are selected from trisodium ethylenediamine disuccinate, ethylenediaminetetraacetic acid (EDTA), tetrasodium glutamate diacetate, tetrasodium etidronate, tetrasodium pyrophosphate, pentasodium ethylenediamine tetramethylene phosphonate, sodium staminate and combinations of these.

Thus, one or a combination of chelating agents may be present, by weight, based on the total weight of the fluid emulsion cosmetic composition, each one or the combination present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 up to about 2 weight percent, including increments and ranges therein and there between.

### Fillers (Including Powders)

In accordance with some embodiments, the fluid emulsion cosmetic composition may comprise one or more fillers. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

In some particular embodiments, the fluid emulsion cosmetic composition may include fillers in the form of powders. In some embodiments, the fluid emulsion cosmetic composition includes at least one powder selected from the group consisting of silica, pigments, boron nitride, lauroyl lysine, perlite, talc, clays, cellulose, and combinations thereof.

More generally, some examples of oil-absorbing fillers include: mica, Zea may (corn) starch, magnesium oxide, nylon-12, nylon-66, cellulose, polyethylene, talc, talc (and) methicone, talc (and) dimethicone, perlite, sodium silicate, pumice, PTFE, Ammonium Polyacryloyldimethyl Taurate, polymethyl methacrylate, Oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxymethyl starch. According to one preferred embodiment, the oil-absorbing filler comprises spherical microparticles of porous silica having a mean particle size from 0.5 to 20 μm whose INCI name is silica sold by the company JCG Catalysts and Chemicals under the name Spheron L-1500. According to another preferred embodiment, the oil absorbing filler comprises hydrophobic aerogel particles whose INCI name is silica silylate sold by Dow Corning under the name VM-2270 Aerogel Fine Particles.

Some examples of optical effects fillers include: bismuth oxychloride, silica silylate, boron nitride, iron oxide, calcium carbonate, calcium sulfate (and) iron oxides, sodium potassium aluminum silicate.

Some examples of fillers which provide both oil-absorbing and optical effects include: silica, silica (and) methicone, silica (and) dimethicone, polysilicone-22, polysilicone-8, polysilicone-11, methyl methacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

The filler, for example a powder, may be present in the fluid emulsion cosmetic composition, at a concentration, or from about 0.01% to 25%, or from about 0.01% to 0.5%, or from about 0.1% to 0.4%, or from about 0.2% to 0.3%, or from about 0.5% to 5.0%, or from about 1.0% to 5.0%, or from about 1.2% to 3.5%, or from about 1.0% to 2.5%, or from about 1.5% to about 2%, or from about 1.0% to about 1.8%, or from about 2.2% to about 2.7% or from about 0.25% to about 0.4%, or any suitable combination, sub-combination, range, or sub-range thereof by weight, based on the weight of the fluid emulsion cosmetic composition. One of ordinary skill in the art, however, will appreciate that other ranges are within the scope of the invention. In some embodiments, the fluid emulsion cosmetic composition includes more than one filler, each filler present in an amount as set forth herein above, wherein each different filler may be present within one of the ranges selected from the ranges set forth herein above.

Thus, each one or combination of filler, when present, may be present by weight, based on the total weight of the fluid emulsion cosmetic composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to about 25.0 percent by weight, including increments and ranges there between.

### Optional Actives and Other Ingredients

In some embodiments, there may be one or more optional actives or other ingredients (herein, “additives”) present in the fluid emulsion cosmetic composition.

In some embodiments, additives may include one or a combination of antimicrobial agents and their salts, for example, including, but not limited to, the group consisting of chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol, and combinations thereof.

In some embodiments, the one or more additives present in the fluid emulsion cosmetic composition may include one or more other components, for example, including, but not limited to, the group consisting of penetrants; sequestrants; fragrances; dispersants; ceramides; opacifiers and combinations thereof. Although the aforementioned optional components are given as an example, it will be appreciated that other optional components compatible with cosmetic applications known in the art may be used.

In some embodiments the composition may include a penetrant or penetration enhancer selected from Ethoxydiglycol, Dimethyl isosorbide, Sodium dilauramidoglutamide lysine, Polyglyceryl Fatty Acid Ester, Laurocapram, dipropylene glycol, propylene glycol, or a combination thereof, may be included.

In accordance with the various embodiments, the amounts of additives, for example, actives and other components, present in the fluid emulsion cosmetic composition can range from about 0.001% to about 50%, from about 0.5% to about 30%, from about 1.5% to about 20%, and from about 5% to about 15%, or any suitable combination, sub-combination, range, or sub-range thereof by weight, based on the weight of the fluid emulsion cosmetic composition.

In some embodiments, one or more additives, alone or in combination, can be present in one or both of the fluid emulsion cosmetic composition from about 0.05% to about 50% by weight, from about 0.05% to about 2.5% by weight, from about 0.1% to about 2%, from about 0.25% to about 1.5%, and from about 0.5% to about 1.25%, or any suitable combination, sub-combination, range, or sub-range thereof by weight, based on the weight of the fluid emulsion cosmetic composition.

In some embodiments, one or more other components, such as preservatives, vitamins, preservatives, and the like, alone or in combination, can be present in the fluid emulsion cosmetic composition from about 0.05% to about 50% by weight, from about 0.05% to about 25% by weight, from about 0.1% to about 10%, from about 0.25% to about 5%, and from about 0.5 to about 3.5%, or any suitable combination, sub-combination, range, or sub-range thereof by weight, based on the weight of the fluid emulsion cosmetic composition.

Thus, one or a combination of additives may be present in the fluid emulsion cosmetic composition, by weight, based on the weight of the fluid emulsion cosmetic composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### EXAMPLES

### Example 1: Raw Materials

The following raw materials were used in the examples herein.

TABLE 1
Raw Materials

| | Commercial | Percent |
|---|---|---|
| Raw Material | Source | Active |
| acrylates/C10-30 alkyl | Pemulen EZ4U | 100% |
| acrylate crosspolymer | | |
| Laureth-4 | | 100% |
| PEG-7 glyceryl cocoate | | 100% |
| oleth-10 | | 100% |
| oleth-5 | | 100% |
| lareth-9 | | 100% |
| PEG-7 caprylic/capric glycerides | | 100% |
| Polyacrylate Crosspolymer-6 | Sepimax Zen | |
| Hydroxyethyl Acrylate/Sodium | EMT-10 | |
| Acryloyldimethyl Taurate | | |
| Copolymer | | |

### Example 2: Representative Compositions

TABLE 2
Inventive and Comparative Compositions

| INCI US | INV 1 | INV 2 | BASE | COMP 1 | COMP 2 | COMP 3 |
|---|---|---|---|---|---|---|
| Actives* | | ~15 | ~15 | ~15 | ~15 | |
| Actives** | ~15 | | | | | ~15 |
| Water soluble solvents | ~12 | ~12 | ~12 | ~12 | ~12 | ~12 |
| (GLYCERIN, ALCOHOL | | | | | | |
| DENAT., BUTYLENE | | | | | | |
| GLYCOL, PENTYLENE | | | | | | |
| GLYCOL | | | | | | |
| WATER (~66-68) | QS | QS | QS | QS | QS | QS |
| ACRYLATES/C10-30 | 0.150 | 0.150 | 0.150 | 0.150 | 0.150 | 0.150 |
| ALKYL ACRYLATE | | | | | | |
| CROSSPOLYMER | | | | | | |
| XANTHAN GUM | 0.350 | 0.350 | 0.350 | 0.350 | 0.350 | 0.350 |
| LAURETH-4 | 0.500 | | | | | |
| PEG-7 GLYCERYL | | 0.500 | | | | |
| COCOATE | | | | | | |
| POLYGLYCERYL-4 | 0.500 | 0.500 | 0.500 | 1.000 | 0.500 | 0.500 |
| CAPRATE | | | | | | |
| POLYGLYCERYL-6 | | | | | | 0.500 |
| DICAPRATE | | | | | | |
| POLYGLYCERYL-2 | | | | | 0.500 | |
| ISOSTEARATE | | | | | | |
| TRISODIUM | 0.300 | 0.300 | 0.300 | 0.300 | 0.300 | 0.300 |
| ETHYLENEDIAMINE | | | | | | |
| DISUCCINATE | | | | | | |
| PHENOXYETHANOL | 0.500 | 0.500 | 0.500 | 0.500 | 0.500 | 0.500 |
| PENTAERYTHRITYL | ~4 | ~4 | ~4 | ~4 | ~4 | ~4 |
| TETRAISOSTEARATE | | | | | | |
| (~0.8), SQUALANE | | | | | | |
| (~2.2), C15-19 ALKANE | | | | | | |
| (~1) | | | | | | |
| Pigment (RED 4, RED 33) | >0.002 | >0.002 | >0.002 | >0.002 | >0.002 | >0.002 |

*Actives: sodium thiosulfate, arginine, sodium hyaluronate, dipotassium glycyrrhizate, capryloyl salicylic acid, carnosine, hydroxyacetophenone, niacinamide, citric acid, caffeine, glycine-based skin conditioning agent or
**without carnosine

### Example 3: Demonstration of Effect of Ethoxylated Agents Vs Other Emulsifiers on Stability

To demonstrate the effect of Ethoxylated Agents on stability, a composition base, BASE, was prepared. This composition is an oil in water emulsion serum that contains acrylates/C10-30 alkyl acrylate crosspolymer at 0.15% as the stabilizing polymer, with xanthan gum as thickener to increase viscosity. It also contains about 12% total actives, a penetration enhancer, preservatives, solvents, and 4% total oil phase. These ingredients all remain constant in all formulations tested for the effects of ethoxylated and other agents on stability, in particular, freeze-thaw.

In this protocol, which represents potential conditions during shipping and storage, one ounce of each tested composition is dispensed in a jar that is placed in a chamber that cycles from −20° C. to 20° C. each 24-hour period, and back, and repeats daily. Samples are observed after 5 cycles (days) and 10 cycles (days). Notably, the base composition does not pass freeze-thaw stability.

From this BASE, 0.5% of each of different emulsifiers were added to determine if their addition improved stability. The examples are outlined below and correspond to the formulations shown in Table 2 herein above.

TABLE 3
Test formulations and HLB

| Tested Composition | BASE+ | HLB |
|---|---|---|
| INV 1 | 0.5% laureth-4 | 10 |
| INV 2 | 0.5% PEG-7 glyceryl cocoate | 11 |
| COMP 1 | 0.5% polyglyceryl-4 caprate | 14 |
| COMP 2 | 0.5% polyglyceryl-2 diisostearate | 5 |
| COMP 3 | 0.5% polyglyceryl-6 dicaprate | 10 |

The formulas INV 1 and INV 2 include ethoxylated agents, while the three comparatives, COMP 1, COMP 2, and COMP 3, INCLUDE emulsifiers that are oligomeric in nature, they are formed using repeat units of glycerin, as opposed to repeat united of ethylene oxide.

All emulsifiers are liquid at room temperature; the composition can be made without addition of heat.

TABLE 4
Test results

| Formula | Emulsifier | RESULTS |
|---|---|---|
| BASE | Base; none | Passes T0; instability increases over time; |
| | | unacceptable at 5 days and 10 days |
| INV 1 | laureth-4 | Passes all timepoints; no difference in |
| | | emulsion quality |
| INV 2 | PEG-7 glyceryl | Passes T0 and 5 days; less stable at 10 days |
| | cocoate | but better than base and other formulations |
| | | below |
| COMP 1 | polyglyceryl-4 | Passes T0; instability increases over time; |
| | caprate | unacceptable at 5 days and 10 days |

The base with added ethoxylated agents showed greatly improved freeze/thaw stability verses the base and the polyglyceryl agents as shown by microscope image analysis.

It was found that though laureth-4, PEG-7 glyceryl cocoate, and polyglyceryl-6 dicaprate all have very similar HLB values, only laureth-4 and PEG-7 glyceryl cocoate showed a large improvement in stability. Therefore, the HLB value is not the main driver of the stability, which is an unexpected attribute of this system.

In accordance with some embodiments, it is contemplated that some or all of following ingredients may be excluded from the fluid emulsion cosmetic composition: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; sucrose stearate and other emulsifiers based on sugar esters; glyceryl stearate and other glycerol esters; disodium ethylene dicocamide PEG-15 disulfate; sodium stearoyl glutamate and other fatty amides; steareth-100 and other fatty ethers, cationic surfactants, and other cationic compounds; silicones, such as silicone polymers, silicone elastomers, and silicone oils, for example selected from dimethicone, cyclomethicone, polysilicone-11, phenyl trimethicone, trimethylsilylamodimethicone, stearoxytrimethylsilane, cyclopentasiloxane, cyclohexasiloxane, and combinations thereof; phthalates, parabens, sulfates, polyquaternium, microplastics, synthetic dyes, gelling agents, and combinations thereof.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A fluid emulsion cosmetic composition comprising: a direct oil in water emulsion comprising at least one synthetic polymer or crosspolymer, at least one thickening polysaccharide, at least one ethoxylated agent having an HLB in a range from 8-14 and in a liquid form at room temperature, at least one active selected from water soluble skin care actives, oil soluble skin care actives, and combinations thereof, water, and at least one oil, wherein the composition is free of silicones.

2. The fluid emulsion cosmetic composition according to claim 1, wherein the fluid emulsion cosmetic composition has a serum galenic that is stable from emulsion breaking and separation under freeze thaw conditions.

3. The fluid emulsion cosmetic composition according to claim 1, wherein the fluid emulsion cosmetic composition maintains a fluid texture that does not break, become creamy or waxy or gellify, including over freeze thaw cycles, retaining a combination of water soluble and oil soluble actives present in an amount that is at least 10% by weight of the composition.

4. The fluid emulsion cosmetic composition according to claim 1, wherein the combination of polymeric thickeners in the fluid emulsion cosmetic composition includes acrylates/C10-30 alkyl acrylate crosspolymer and xanthan gum.

5. The fluid emulsion cosmetic composition according to claim 1, wherein the synthetic polymer or crosspolymer is present in a range from about 0.15% to about 1.5%, at least one thickening polysaccharide is present in a range from about 0.05% to about 2%.

6. The fluid emulsion cosmetic composition according to claim 1, wherein the at least one ethoxylated agent is one of laureth-4 and PEG-7 glyceryl cocoate.

7. The fluid emulsion cosmetic composition according to claim 1, wherein the at least one ethoxylated agent is one of laureth-4 and PEG-7 glyceryl cocoate, each present in a range from about 0.2% to about 5%, by weight of the fluid emulsion cosmetic composition.

8. The fluid emulsion cosmetic composition according to claim 1, wherein actives may include sodium thiosulfate, arginine, sodium hyaluronate, dipotassium glycyrrhizate, capryloyl salicylic acid, carnosine, hydroxyacetophenone, niacinamide, citric acid, caffeine, a glycine-based skin conditioning agent, or a combination thereof, each present in a range from about 0.05% to about 10%, by weight of the composition.

9. The fluid emulsion cosmetic composition according to claim 8, wherein the actives include a combination of water soluble and oil soluble actives, the combination present in an amount that is at least 10% by weight of the composition.

10. The fluid emulsion cosmetic composition according to claim 1, wherein water is present in a range from about 60% to about 70%, by weight of the composition, and the fluid emulsion cosmetic composition includes water-soluble solvents that include glycerin, alcohol denatured, butylene glycol, and pentylene glycol, each present in a range from about 2% to about 4%, by weight of the composition.

11. The fluid emulsion cosmetic composition according to claim 1, wherein the at least one oil is selected from the group consisting of pentaerythrityl tetraisostearate, c15-19 alkane, squalane, and combinations thereof.

12. The fluid emulsion cosmetic composition according to claim 1, wherein the at least one oil is present in a range from about 0.5% to about 3, by weight of the fluid emulsion cosmetic composition.

13. The fluid emulsion cosmetic composition according to claim 1, wherein the at least one oil includes a combination of pentaerythrityl tetraisostearate, c15-19 alkane, and squalane, each present in a range from about 0.5% to about 3, by weight of the fluid emulsion cosmetic composition.

14. The fluid emulsion cosmetic composition according to claim 1, wherein the at least one synthetic polymer or crosspolymer is present in a range from about 0.15% to about 1.5%, the at least one thickening polysaccharide is present in a range from about 0.05% to about 2%, the at least one ethoxylated agent having an HLB in a range from 8-14 is present in a range from about 0.5% to about 5%, each active is present in a range from about 1% to about 20%, the at least one oil is present in a range from about 2% to about 4%, all amounts by weight, based on the weight of the fluid emulsion cosmetic composition.

15. The fluid emulsion cosmetic composition according to claim 1, wherein the least one synthetic polymer or crosspolymer is selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof, the least one thickening polysaccharide is selected from the group consisting of Sclerotium gum, xanthan gum, cellulose gum, locust bean gum, carrageenan, and combinations thereof, the at least one ethoxylated agent having an HLB in a range from 8-14 is selected from laureth-4 and PEG-7 glyceryl cocoate, the at least one active is selected from water soluble skin care actives, oil soluble skin care actives, and combinations thereof, water, and the at least one oil is selected from the group consisting of pentaerythrityl tetraisostearate, c15-19 alkane, squalane, and combinations thereof.

16. The fluid emulsion cosmetic composition according to claim 15, wherein the actives include a combination of water soluble and oil soluble actives, the combination present in an amount that is at least 10% by weight of the composition.

17. A fluid emulsion cosmetic composition comprising: a direct oil in water emulsion comprising at least one synthetic polymer or crosspolymer comprising acrylates/C10-30 alkyl acrylate crosspolymer, at least one thickening polysaccharide comprising xanthan gum, at least one ethoxylated surfactant in a liquid form at room temperature and comprising one of laureth-4 and PEG-7 glyceryl cocoate, a combination of water soluble and oil soluble actives comprising sodium thiosulfate, arginine, sodium hyaluronate, dipotassium glycyrrhizate, capryloyl salicylic acid, carnosine, hydroxyacetophenone, niacinamide, citric acid, caffeine, a glycine-based skin conditioning agent, water and water soluble solvents comprising glycerin, alcohol denatured, butylene glycol, and pentylene glycol, and a combination of oils comprising pentaerythrityl tetraisostearate, c15-19 alkane, and squalane, wherein the composition is free of silicones.

18. The fluid emulsion cosmetic composition according to claim 17, wherein the fluid emulsion cosmetic composition maintains a fluid texture that does not break, become creamy or waxy or gellify, including over freeze thaw cycles, retaining the combination of water soluble and oil soluble actives present in an amount that is at least 10% by weight of the composition.

19. A method of forming a fluid emulsion cosmetic composition as a direct oil in water emulsion, the method comprising providing at least one synthetic polymer or crosspolymer, at least one thickening polysaccharide, at least one ethoxylated agent having an HLB in a range from 8-14 that is liquid at room temperature, at least one active selected from water soluble skin care actives, oil soluble skin care actives, and combinations thereof, water, and at least one oil, wherein the composition is free of silicones, wherein the method includes blending all ingredients at room temperature without the application of heat.

20. The method according to claim 19, wherein the actives include a combination of water soluble and oil soluble actives, the combination present in an amount that is at least 10% by weight of the composition.
